(19) United States
(12) Patent Application Publication
(10) Pub. No.: US 2012/0021453 A1
(43) Pub. Date: Jan. 26, 2012

(54) LABEL-FREE CELL SORTING USING NEAR INFRARED EMISSION

(75) Inventors: Hirak Patra, Andharia (IN); Anjan Kr. Dasgupta, Kolkata (IN)

(73) Assignee: University of Calcutta, Kolkata, West Bengal (IN)

(21) Appl. No.: 13/123,209

(22) PCT Filed: Aug. 6, 2010
(86) PCT No.: PCT/IB2010/001954

§ 371 Date: Apr. 7, 2011

(30) Foreign Application Priority Data
Apr. 26, 2010 (IN) 467/KOL/2010

(51) Int. Cl. C12Q 1/04

(52) U.S. Cl. 435/34

(57) ABSTRACT

Disclosed are methods and systems for identifying and sorting cells based on a near-infrared emission pattern of the cell in response to excitation at 630±nm. The NIR emission pattern can be used for monitoring and sorting of cells in a label-free manner, and thus provides a positive method for selecting cells, such as stem cells, for use in therapy.

### TECHNICAL FIELD

The present technology relates generally to the field of flow cytometry. More specifically, it relates to identifying spectral patterns that are associated with particular cell types.

### BACKGROUND

The following description is provided to assist the understanding of the reader. None of the information provided or references cited is admitted to be prior art.

Flow cytometry is a technique for counting and examining microscopic particles, such as cells, by suspending them in a stream of fluid and passing them by a detection apparatus. Flow cytometry is routinely used in the diagnosis of health disorders, especially blood cancers, but has many other applications in both research and clinical practice. A common variation is to physically sort particles based on their properties, so as to purify cell populations of interest. In positive selection techniques, the desired cells are labeled with antibodies and removed from the remaining unlabeled/unwanted cells. In negative selection, the unwanted cells are labeled and removed.

Stem cells are undifferentiated cells. They retain the ability to divide throughout life and give rise to both new stem cells and to more differentiated/specialized cells which can take the place of cells that die or are lost. Thus, stem cells contribute to the body's ability to renew and repair its tissues, because unlike mature (differentiated) cells, they are not permanently committed to their fate. Stem cells are recognized as being “multipotent” or “pluripotent”, i.e. as having the ability to differentiate into more than one type of specialized mature cell. “Adult stem cells” are cells with these characteristics that are derived from non-embryonic sources. This can include neonates, older individuals, and umbilical cord blood. Other terms for “adult stem cells” include tissue stem cells, somatic stem cells and post-natal stem cells.

Adult stem cells may arise from many different tissue types. Studies have identified bone marrow stem cells, peripheral blood stem cell, neuronal stem cells, muscle stem cells, liver stem cells, pancreatic stem cells, corneal limbal stem cells, mammary stem cells, salivary gland stem cells, stomach stem cells, skin stem cells, tendon stem cells, synovial membrane stem cells, heart stem cells, cartilage stem cells, thymic progenitor stem cells, dental pulp stem cells, adipose derived stem cells, umbilical cord blood and mesenchymal stem cells, amniotic stem cells, mesangioblasts, and colon stem cells. Because many adult stem cells are multipotent but not pluripotent, exploitation of adult stem cells may depend on the ability to readily identify and isolate stem cells of different types. Identification of cells as stem cells typically relies on the use of cell surface markers or cellular differentiation (CD) antigens as indicators of the genomic activity related to a particular differentiation state, or the absence of indicators of more differentiation (such as expression of specialized enzymes).

Stem cells may be useful in a variety of therapies. However, contamination of stem cells by other cell types during transplantation may generate an undesirable toxic response in the host (particularly in case of allogenic transplantation). Sorting can be performed using techniques like flow cytometry only if stem cell specific labels are employed. Though accepted stem cell markers are available, the use of these markers prior to transplantation is not practical because the label may interfere with the activity of the cell. Only negative selection (for example, elimination of non-stem cells by centrifugation) is feasible. However, this is much less efficient than positive selection because some percentage of stem cells are pelleted and eliminated prior to the transplant. Major cases of mortality in stem cell transplants originate from complications related to transplantation and graft failure which are in turn related to a low stem cell population remaining after negative selection. Real-time monitoring and enrichment of stem cells prior to the transplantation may improve the success of these procedures.

### SUMMARY

In one aspect, the present disclosure provides a method for identifying one or more cells in a sample, the method comprising: passing a cell from the sample through a cell detection zone; illuminating the cell in the cell detection zone with an effective amount of electromagnetic radiation to produce a near-infrared emission; and analyzing an intensity or pattern of the near-infrared emission to identify the cell in the cell detection zone. In one embodiment, the electromagnetic radiation is produced by a laser. In one embodiment, a wavelength of electromagnetic radiation produced by the laser is about 630±20 nm. In one embodiment, the near-infrared emission is about 900 to about 1000 nm. In one embodiment, there is a first near-infrared emission at about 900-910 nm and a second near-infrared emission at about 960 nm.

In one embodiment, the cell is a eukaryotic cell, a prokaryotic cell, an embryonic stem cell, or an adult stem cell. In one embodiment, the cell is a red blood cell, a platelet, a mononuclear cell, an embryonic stem cell, an adult stem cell, or a hematopoietic stem cell. In one embodiment, the sample comprises at least one cancer cell. In one embodiment, the analyzing comprises detecting the at least one cancer cell. In one embodiment, the analyzing comprises detecting cancer cells that have been exposed to different anti-cancer agents. In one embodiment, the analyzing comprises comparing the near-infrared emission pattern to a near-infrared emission pattern of a normal cell, or to a near-infrared emission of a cancer cell, or to both, in order to detect the at least one cancer cell. In one embodiment, the sample contains or is suspected to contain at least one pathogen. In one embodiment, the analyzing comprises detecting the at least one pathogen, if present.

In one embodiment, the method further comprises detecting one or more additional label-free characteristics of the cell. In one embodiment, the one or more additional label-free characteristics of the cell are selected from the group consisting of: forward scattering, side scattering, and pseudo-Raleigh scattering (occurring at twice the excitation wavelength).

In one embodiment, passing the cell from the sample through the cell detection zone is by flow cytometry. In one embodiment, the methods further comprise sorting the cell. In one embodiment, sorting comprises removing cells that are not in a cell population of interest. In one embodiment, the cells that are not in the cell population of interest are destroyed.

In one aspect, the disclosure provides a method for enriching a population of a desired cell type, the method comprising: introducing a heterogeneous mixture of cells into a flow stream; passing each cell in the heterogeneous mixture of cells through a cell detection zone; illuminating the cell in the cell detection zone with an effective amount of electromagnetic radiation to produce a near-infrared emission in the cell detection zone; and collecting the cells that have a substantially identical intensity of the near-infrared emission to the desired cell type in order to produce an enriched population of cells.

In another aspect, the disclosure provides a flow cytometer system comprising: a light source capable of producing an effective amount of electromagnetic radiation to produce a near-infrared emission in the sample; a flow chamber that is optically connected to the light source, wherein cells to be detected flow through the flow chamber while being carried by a sheath fluid; and a signal processing unit for collecting and analyzing an emission from the cells in the flow chamber and outputting the results thereof. In one embodiment, the light source is a laser. In one embodiment, a wavelength of light energy produced by the laser is about 630±20 nm.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE FIGURES

FIG. 1 depicts a block diagram of an illustrative embodiment of cell identification system.

FIG. 2 is an illustration of the detection of near-infrared fluorescence (NIRF) from a cell, as well as optional detection of additional label-free parameters, such as forward scattering (FSC), side scattering (SSC), and pseudo-Raleigh scattering.

FIG. 3 is an illustrative embodiment of a cell sorting apparatus using NIRF.

FIG. 4 is a graph illustrating the water NIRF for red blood cells (RBC), mononuclear cells (MNC) and platelets in normal saline. The gray bar indicates the cut-off region in which one expects only the mononuclear cells to be present.

FIG. 5 is an illustrative embodiment showing how different cutoffs in fluorescence intensity can be used to identify different cell populations.

FIG. 6 is an illustrative embodiment showing the integration of NIRF with forward and side scattering in order to sort a cell based on multiple characteristics or dimensions.

FIG. 7 is graph illustrating the survivability of RPMI 8226 cells treated with different agents (VS, GNP, and GNP-VS).

FIG. 8 shows illustrative NIR emission spectra for RPMI 8226 cells treated with different agents (VS, GNP, and GNP-VS).

FIG. 9 is a graph illustrating the effect of two different nanoparticles (GNP-VS) and (R-GNP-VS) on the NIR emission of RPMI 8226 cells.

### DETAILED DESCRIPTION

In the following detailed description, reference may be made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented here.

As used herein, unless otherwise stated, the singular forms “a,” “an,” and “the” include plural reference. Thus, for example, a reference to “a protein” includes a plurality of protein molecules.

As used herein, the term “about” will be understood by persons of ordinary skill in the art and will vary to some extent depending upon the context in which it is used. If there are uses of the term which are not clear to persons of ordinary skill in the art, given the context in which it is used, the term “about” in reference to quantitative values will mean up to plus or minus 10% of the enumerated value.

As used herein, the term “electromagnetic radiation” refers to any type of electromagnetic radiation or energy, whether comprised of a narrow, discrete frequency or multiple frequencies. Examples of electromagnetic radiation include visible light, infrared radiation, and ultraviolet radiation. In one embodiment, the term “electromagnetic radiation” means the energy of rays capable of providing sufficient excitation energy to water in order to induce an emission in the range of about 900 to about 1000 nm.

As used herein, the term “emission” refers to the emission of radiation by a substance that has absorbed light energy of a different wavelength. The emission of this type can be caused by fluorescence in which absorption of a photon triggers the emission of a photon with a longer (less energetic) wavelength. The energy difference between the absorbed and emitted photons ends up as molecular rotations, vibrations or heat, for example. In one embodiment, the emission may be caused by inelastic scattering (e.g., Raman scattering) in which similar Stokes emission may be observed, which co-occurs with an anti-Stokes line.

As used herein, the term “laser” refers to electromagnetic radiation of any frequency that is amplified by stimulated emission of radiation. A laser also refers to a device that emits electromagnetic radiation through a process called stimulated emission. Laser light is usually spatially coherent, which means that the light either is emitted in a narrow, low-divergence beam, or can be converted into one with the help of optical components such as lenses. As used herein, the term “red wavelength laser radiation” refers to laser radiation having wavelengths in the range from about 600 to about 700 nm.

As used herein, the term “substantially pure” or “substantially homogenous” means an object species is the predominant species present (i.e., on a molar basis it is more abundant than any other individual species in the composition). Generally, a substantially pure composition will be more than about 80%, more than about 90%, more than about 95%, more than about 97%, more than about 98%, more than about 99%, or more than about 99.5% of all species present in the composition. Typically, the object species is purified to essential homogeneity (contaminant species cannot be detected in the composition by conventional detection methods) when the composition consists essentially of a single macromolecular species. The term “homogeneous population of cells” refers to a population of cells wherein at least about 80%, or at least about 90%, or at least about 95% of the cells in the population are of the same cell type.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascities fluid, or whole blood, and including biopsy samples of body tissue. A sample may also include an in vitro culture of cells. A sample may be obtained from any subject, e.g., a subject/patient having or suspected to have a disease.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease.

As used herein, the term “substantially identical intensity” means that two or more spectral patterns do not exhibit a statistically significant difference. For example, a difference may be statistically significant if the measured NIRF intensity for a particular cell falls outside of about 1.0 standard deviations, about 1.5 standard deviations, about 2.0 standard deviations, or about 2.5 stand deviations of the mean intensity for any control or reference group.

### Systems for Label-Free Cell Identification

The disclosure is based on the discovery that near-infrared fluorescence (NIRF) of water produces a spectral signature based on a characteristic water nanocluster distribution in a given cell. Without wishing to be limited by theory, this fluorescence has cell-type dependence because water distribution in individual cells depends largely on the specific nanoclusters of water distributed over the cell surface. As such, the NIRF pattern can be used for monitoring and sorting of cells in a label free manner.

In one aspect, the disclosure proves an apparatus for analyzing a cell and obtaining its NIRF spectrum. A system including hardware and software for analyzing the spectrum and characterizing the cell is provided. The apparatus includes light sources, such as a laser, as well as optics and filters to present the laser light to the sample and collect the NIRF signals from the sample. The optics can be fiber optics for increased compactness. The system can also comprise an inverted and phase contrast microscope, CCD camera, compact fiber based spectrometers, computer, software, and a flow cell sample collection system. The computer and the software may be automated to obtain the NIRF spectrum from the sample, perform an analysis on the spectrum, and compare the results to a database to characterize or identify the cell.

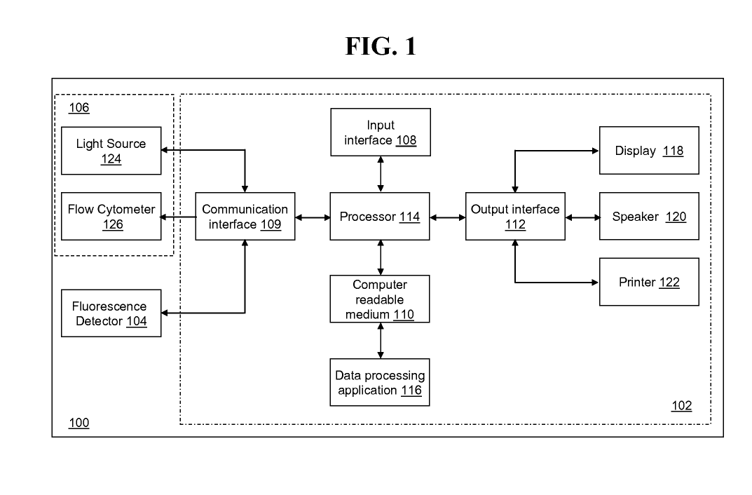

With reference to FIG. 1, a block diagram of a system for identifying cells using NIRF is shown in accordance with an illustrative embodiment. Cell identification system 100 may include one or more of a computing system 102, a fluorescence detector 104, and a sample analysis instrument 106. Different and additional components may be incorporated into cell identification system 100. Computing system 102 may include one or more of an input interface 108, a communication interface 109, a computer-readable medium 110, an output interface 112, a processor 114, a data processing application 116, a display 118, a speaker 120, and a printer 122. Different and additional components may be incorporated into computing system 102.

Input interface 108 provides an interface for receiving information from the user for entry into computing system 102 as known to those skilled in the art. Input interface 108 may use various input technologies including, but not limited to, a keyboard, a pen and touch screen, a mouse, a track ball, a touch screen, a keypad, one or more buttons, etc. to allow the user to enter information into computing system 102 or to make selections presented in a user interface displayed on display 118. The same interface may support both input interface 108 and output interface 112. For example, a touch screen both allows user input and presents output to the user. Computing system 102 may have one or more input interfaces that use the same or a different input interface technology.

Communication interface 109 provides an interface for receiving and transmitting data between devices using various protocols, transmission technologies, and media as known to those skilled in the art. Communication interface 109 may support communication using various transmission media that may be wired or wireless. Computing system 102 may have one or more communication interfaces that use the same or a different communication interface technology. Data and messages may be transferred between computing system 102, fluorescence detector 104, and/or sample analysis instrument 106 using communication interface 109.

Computer-readable medium 110 is an electronic holding place or storage for information so that the information can be accessed by processor 114 as known to those skilled in the art. Computer-readable medium 110 can include, but is not limited to, any type of random access memory (RAM), any type of read only memory (ROM), any type of flash memory, etc. such as magnetic storage devices (e.g., hard disk, floppy disk, magnetic strips, etc.), optical disks (e.g., CD, DVD, etc.), smart cards, flash memory devices, etc. Computing system 102 may have one or more computer-readable media that use the same or a different memory media technology. Computing system 102 also may have one or more drives that support the loading of a memory media such as a CD or DVD. Computer-readable medium 110 may provide the electronic storage medium for fluorescence detector 104 and/or sample analysis instrument 106. Computer-readable medium 110 further may be accessible to computing system 102 through communication interface 109.

Output interface 112 provides an interface for outputting information for review by a user of computing system 102. For example, output interface 112 may include an interface to display 118, speaker 120, printer 122, etc. Display 118 may be a thin film transistor display, a light emitting diode display, a liquid crystal display, or any of a variety of different displays known to those skilled in the art. Speaker 120 may be any of a variety of speakers as known to those skilled in the art. Printer 122 may be any of a variety of printers as known to those skilled in the art. Computing system 102 may have one or more output interfaces that use the same or a different interface technology. Display 118, speaker 120, and/or printer 122 further may be accessible to computing system 102 through communication interface 109.

Processor 114 executes instructions as known to those skilled in the art. The instructions may be carried out by a special purpose computer, logic circuits, or hardware circuits. Thus, processor 114 may be implemented in hardware, firmware, or any combination of these methods and/or in combination with software. The term “execution” is the process of running an application or the carrying out of the operation called for by an instruction. The instructions may be written using one or more programming language, scripting language, assembly language, etc. Processor 114 executes an instruction, meaning that it performs/controls the operations called for by that instruction. Processor 114 operably couples with input interface 108, with communication interface 109, with computer-readable medium 110, and with output interface 112, to receive, to send, and to process information. Processor 114 may retrieve a set of instructions from a permanent memory device and copy the instructions in an executable form to a temporary memory device that is generally some form of RAM. Computing system 102 may include a plurality of processors that use the same or a different processing technology.

Data processing application 116 performs operations associated with processing data for a sample gathered using one or more electronic devices that continuously, periodically, and/or upon request monitor, sense, measure, etc. the physical and/or chemical characteristics of the sample. The operations may be implemented using hardware, firmware, software, or any combination of these methods. With reference to the illustrative embodiment of FIG. 1, data processing application 116 is implemented in software (comprised of computer-readable and/or computer-executable instructions) stored in computer-readable medium 110 and accessible by processor 114 for execution of the instructions that embody the operations of data processing application 116. Data processing application 116 may be written using one or more programming languages, assembly languages, scripting languages, etc.

Fluorescence detector 104 may include a fluorescence detection system such as a fluorometer, etc. Fluorescence detector 104 generates data related to a sample, such as the intensity of NIRF from the sample. The source of and the dimensionality of the data is not intended to be limiting. Computing system 102 may be separate from or integrated with fluorescence detector 104 to control the operation of fluorescence detector 104.

Sample analysis instrument 106 may include n light source 124 as part of a flow cytometer 126. Different and additional components may be incorporated into sample analysis instrument 106. Light source 124 produces sufficient light energy to generate a near-infrared fluorescence pattern that is characteristic of cells in the sample. Flow cytometer 126 allows for the NIRF of cells to be detected individually.

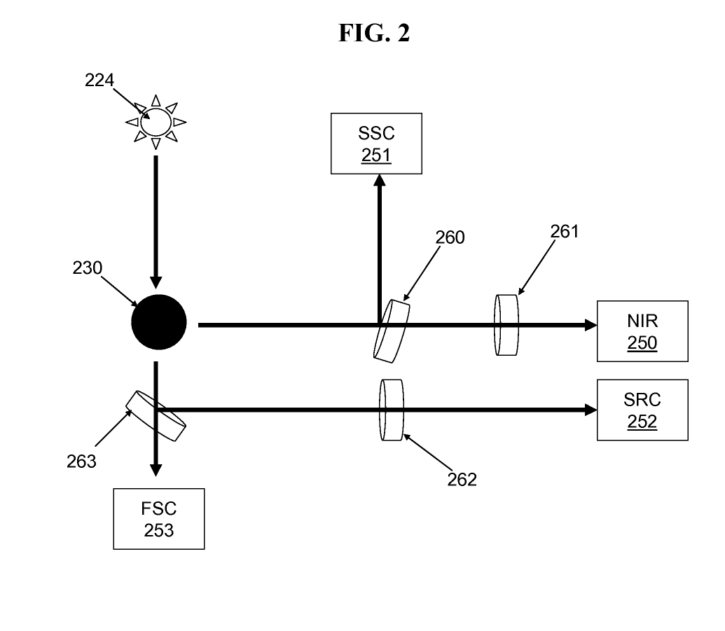

With reference to FIG. 2, a schematic diagram of a system for identifying cells using NIRF and other label-free methods is shown in accordance with an illustrative embodiment. Light source 224 produces sufficient light energy to generate a near-infrared fluorescence pattern that is characteristic of a cell 230 in the sample. A number of detectors 250, 251, 252, 253 may be included. NIRF detector(s) 250 may be used to detect fluorescence emission at about 900 nm and about 960 nm. An optional FSC detector 253 is located at about 2°-16° to the laser light beam and may be used to detect forward scattering. The term FSC as used herein refers to light scattered at angles which can be used primarily to count particles. The lower limit on the FSC angle is determined by the incident beam shape and size. The FSC detector 253 is typically preceded by a beam stop 263, such as an obscuration bar, to prevent the incident beam from directly striking the detector 253. The collection optics can include various wavelength filters 260, 261, 262.

An optional SSC detector 251 may be used to detect side scattering. The term SSC is used herein for angles which, in combination with the FSC signal, can be used primarily to distinguish granular from agranular particles. The SSC angle is an angle providing information about internal structure of particles as shown by their light scattering properties.

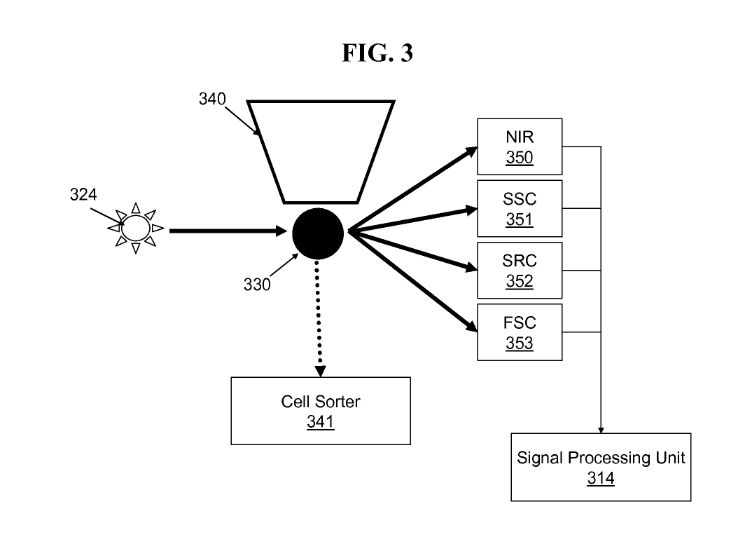

With reference to FIG. 3, a schematic diagram of a system for identifying cells using NIR and other label-free methods is shown in accordance with an illustrative embodiment. Light source 324 produces sufficient light energy to generate a NIR pattern that is characteristic of a cell 330 in the sample that has passed through the nozzle 340 of a flow cytometer (not shown). A number of detectors 350, 351, 352, 353 may be included. NIR detector(s) 350 may be used to detect fluorescence emission at about 900 and about 960 nm. Each of the detectors 350, 351, 352, 353 may communicate with a signal processing unit 314. Cell sorter 341 may be used to sort different cell types in individual sample collectors (not shown).

In one embodiment, the light source for use with the methods will avoid damage to biological materials, such as cells. By choosing wavelengths in ranges where the absorption by cellular components is minimized, the deleterious effects of heating can be avoided. However, a light having a wavelength generally considered to be damaging to biological materials can be used, such as where the illumination is for a short period of time and where deleterious absorption of energy does not occur. In some embodiments, the light sources will be coherent light sources. Typically, the coherent light source will be a laser. However, non-coherent sources may be utilized. Furthermore, if there is more than one light source in the system, these sources can be coherent or incoherent with respect to each other.

In some embodiments, NIRF can be induced by laser radiation operating at a wavelength from about 550 nm to about 750 nm, from about 575 nm to about 725 nm, from about 600 to about 700 nm, or from about 600 to about 650 nm. In an illustrative embodiment, NIRF can be induced by laser radiation operating at a wavelength of about 630±10 nm. In some embodiments, NIRF can be induced by laser radiation operating at a wavelength selected to induce fluorescence of water at from about 950 nm to about 1000 nm or from about 890 to about 910 nm. In an illustrative embodiment, NIRF can be induced by laser radiation operating at a wavelength selected to induce a NIR emission at about 900 nm or about 960 nm.

In some embodiments, NIRF can be induced by low intensity red laser radiation having a power density less than about 10 watt/cm², less than about 5 watt/cm², less than about 4 watt/cm², less than about 3 watt/cm², less than about 2 watt/cm², less than about 1.8 watt/cm², or less than about 1.5 watt/cm². In some embodiments, NIRF can be induced by low intensity red laser radiation having a power density at least about 0.1 watt/cm², at least about 0.2 watt/cm², at least about 0.3 watt/cm², at least about 0.4 watt/cm², at least about 0.5 watt/cm², or at least about 0.6 watt/cm² While the embodiments herein are not limited to the use of a laser, the NIRF effect appears to be most pronounced using laser light.

### Methods for Label-Free Cell Identification and Sorting

In one aspect, the present disclosure provides a method to identify and sort cells, such as stem cells. In some embodiments, the methods are positive selection methods that are based on the NIRF effect of water. These methods provide an advantage over conventional cell sorting methods that are based on negative selection because negative selection methods, such as centrifugation, do not efficiently recover all of the cells of interest. Moreover, the present methods provide an advantage over cell sorting methods that rely on labels, which exert stress on the cells. As such, the present methods provide greater numbers of enriched cells with higher purity than conventional methods.

In one embodiment, a single cell or a collection of cells can be analyzed. An illustrative system is shown in FIGS. 1 and 2 (described above). The cell can be a single cell organism, such as a bacterium, a yeast, and the like, or it can be obtained from a subject such as a human, plant, fish, animal, and the like. The cells from the subject can include, but are not limited to, a normal cell, a cancer cell, a fetal stem cell, an adult stem cell, an activated B or T cell, or a dendritic cell. In an illustrative embodiment, hematopoietic stem cells are identified and isolated by the present methods and used in bone marrow transplantation therapies.

In one embodiment, a NIRF spectra for a plurality of cells can be obtained in order to generate a database of spectra. The NIRF spectra of the plurality of the cells can be averaged to provide a mean NIRF intensity for the cell type. Once a reference spectrum has been obtained for a particular cell type, that spectrum can be compared to spectra from unknown cell types in order to identify the unknown cells. Statistical methods can be used to set thresholds for determining when the NIRF intensity of a cell in an unknown sample can be considered to be different than or similar to a reference level. In addition, statistics can be used to determine the validity of the difference or similarity observed between an unknown intensity level and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between samples deemed significant if the p value is less than or equal to 0.05.

In one aspect, the present methods may be used to sort a heterogeneous population of cells into its constituent cell types. Thus, a substantially homogenous cell population of interest can be obtained. In an illustrative embodiment, the cell population of interest is a population of hematopoietic stem cells. The cells that are not in the population of interest can be destroyed. For example, the laser used for NIRF detection can also be used to kill the cell, such as, for example, by increasing the power output, changing the wavelength of the laser where it is lethal to the cell, and the like. In another aspect, the cells that are not in the population of interest can be sorted from the other cells, similar to fluorescence flow cytometry. For example, after NIRF analysis, the laser can be used to push the normal cells into a container for the cells of interest, while the other cells can be pushed into a separate container.

In one aspect, the present methods are used to isolate substantially homogenous populations of adult stem cells for use in therapy. In one embodiment, adult hematopoietic stem cells (HSCs) are isolated. HCSs are mesenchymal stem cells (MSC) that have the ability to differentiate into various tissue cells. These cells can be found in peripheral blood. In another embodiment, stem cells are isolated from umbilical cord blood from a newborn. The cord blood material is usually discarded at birth, however, cord blood can be used for either autologous or allogenic stem cell replacement. Enrichment of the cord blood stem cells by the characteristic NIRF pattern, and sorting based on the analysis, allows for a smaller amount of material to be stored, which can be more easily given back to the patient or another host. In yet another embodiment, adult stem cells are isolated from various organs. For example, adult stem cells from heart, liver, neural tissue, bone marrow, and the like, have small subpopulations of immortal stem cells which may be manipulated ex vivo and then can be reintroduced into a patient in order to repopulate a damaged tissue. The methods described above can be used to enrich these extremely rare adult stem cells so that they may be used for cell therapy applications.

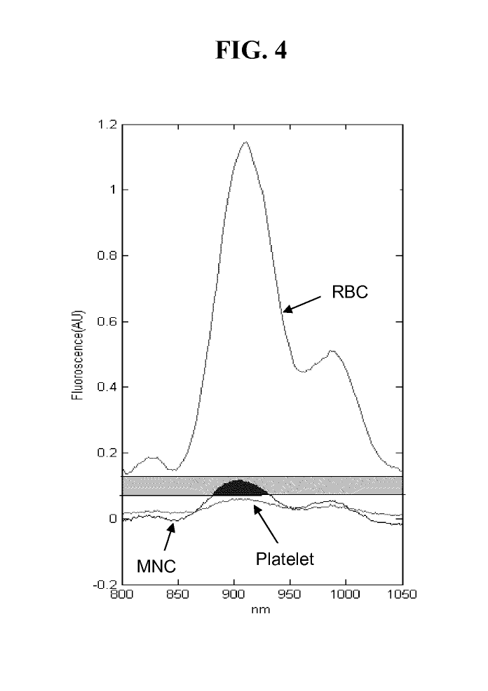

In an illustrative embodiment, the methods include a positive selection process for enriching and recovering human hematopoietic progenitor cells and stem cells in a sample containing human hematopoietic differentiated, progenitor, and stem cells comprising introducing a heterogeneous mixture of cells into a flow stream; passing each cell in the heterogenous mixture of cells through a cell detection zone; and recovering a cell preparation which is enriched in human hematopoietic progenitor cells and stem cells. FIG. 4 shows an illustrative embodiment of how different cell types provide different NIRF signals. Thus, different cutoffs of NIRF intensity can be used in flow cytometry to sort cells into different populations.

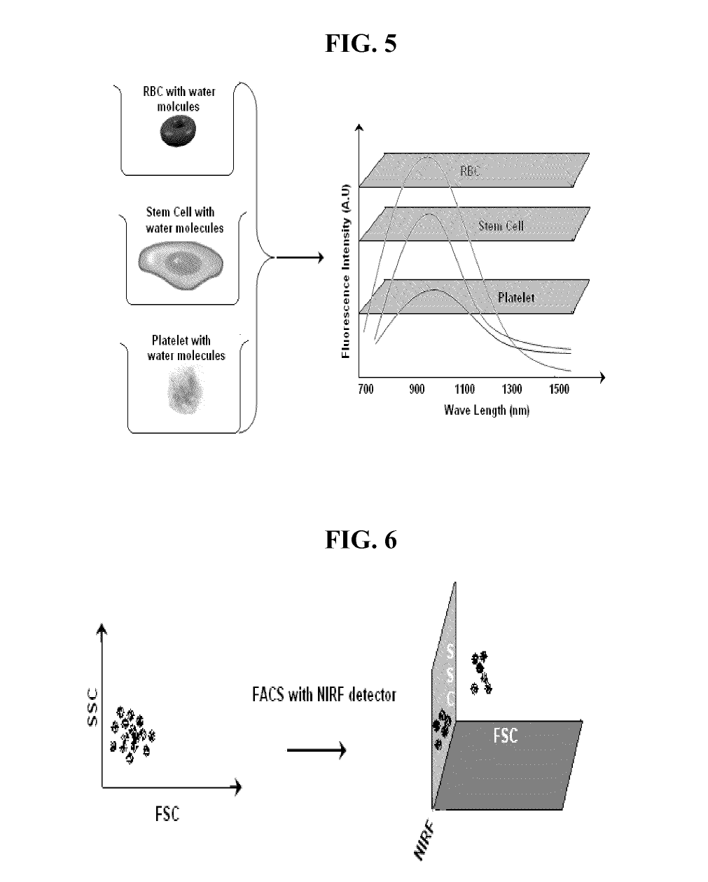

In one embodiment, NIRF intensity is combined with other optical characteristics of cells in order to enhance the identification and sorting of cell populations. In one embodiment, the additional optical characteristics are selected from the group consisting of: forward scattering, side scattering, and Raleigh scattering. FIG. 5 shows an illustrative embodiment of the integration of NIRF with forward and side scattering. These characteristics can be plotted in three dimensions to definitively identify a cell. The difference between the present methods and conventional flow cytometry is that the addition of NIRF allows one to add more dimensions, such as SSC, FSC, and pseudo Raleigh scattering to make the segregation of cell populations more effective.

In one embodiment, the present methods are used to detect diseased cells, such as cancer cells, in a sample. In one embodiment, the diseased cells include blood cell malignancies. Some representative blood cell malignancies include lymphomas, leukemias, and myelomas. Other blood cell malignancies are known in the art. For example, a blood sample may be obtained from a patient having or suspected of having a blood cell disorder. The sample from the patient may be analyzed by flow cytometry in which each cell in the sample of cells passes through a cell detection zone and is illuminated with an effective amount of electromagnetic radiation to produce a near-infrared fluorescence of water in the cell detection zone. The NIRF spectrum of the cell is then compared to a database of spectra to determine if the identified pattern is substantially similar to or different from the known spectra of malignant cells. The presence of malignant cells in a sample may aid in the diagnosis of blood cell disorders.

In another aspect, the methods described above can be used for the detection, identification and/or quantification of single cell organisms, such as, for example, bacteria, yeast, and the like. In particular, the methods can be used for the detection of organisms of specific bacterial genus, species or serotype, in isolated form or as contaminants in environmental or forensic samples, or in foodstuff. A wide variety of single cells can be assessed with these methods. These include for example gram-positive bacteria, gram-negative bacteria, fungi, viruses, etc. Thus, the methods described above can be used to identify pathogens, including, but not limited to, Staphylococcus aureus, Listeria monocytogenes, Bacillus cereus, Salmonella, Cholera, Campylobacter jejuni, and E. coli. It will be seen by those skilled in the art however that other types of cells can be identified using the methods described above.

The detection of single cell organisms can be used, for example, for an early diagnosis of patients suffering from a pathogen infection. Thus, according to the present methods, there is provided a process for the detection of pathogens in the blood, such as bacteria, fungi and viruses, wherein the pathogen is separated using flow cytometry, and the pathogen is detected using NIRF analysis. Further, the harmful cell, upon its detection, can be selectively destroyed. For example, the laser used for NIRF detection can be used to kill the harmful cell, such as, for example, by increasing the power output, changing the wavelength of the laser where it is lethal to the harmful cell, and the like. In another embodiment, the harmful (pathogenic) cells can be sorted from the normal cells, similar to fluorescence flow cytometry. The methods described above can be used to indicate the presence of microbes responsible for disease, and if present, the harmful bacteria can be destroyed.

### EXAMPLES

The present compositions, methods and kits, thus generally described, will be understood more readily by reference to the following examples, which are provided by way of illustration and are not intended to be limiting of the present methods and kits.

### Example 1

### Cell Sorting of Hematopoietic Cells Using NIRF

In this example, the NIRF of cell suspensions of red blood cells (RBC), mononuclear cells (MNC), and platelets in saline were analyzed. The results are shown in FIG. 4. Each population of cells gave a characteristic fluorescence intensity at approximately 910 nm. The figure shows that it is possible to perform single cell sorting (e.g. flow cytometry) in which cells can be sorted on the basis of NIRF intensity. The gray patch shows the cut off region in which one expects only the mononuclear cells which are rich in stem cells. Thus, the NIRF, alone or in combination with FSC and SSC can be used to separate MNCs.

### Example 2

RPMI 8226 human myeloma cells were cultured in normal growth condition and appropriate medium in a tissue culture incubator. The cells are then subjected for the treatment with gold nanoparticles (GNP) at final concentration of 125 μM, Vincristine sulphate (VS) with a final concentration of 10 ng/ml, or gold nanoparticles conjugated with vincristine sulphate at the same concentrations (GNP-VS) for 72 hours. The control and differentially treated cells were then studied using near infrared fluorescence and the standard MTT assay to check the cell viability.

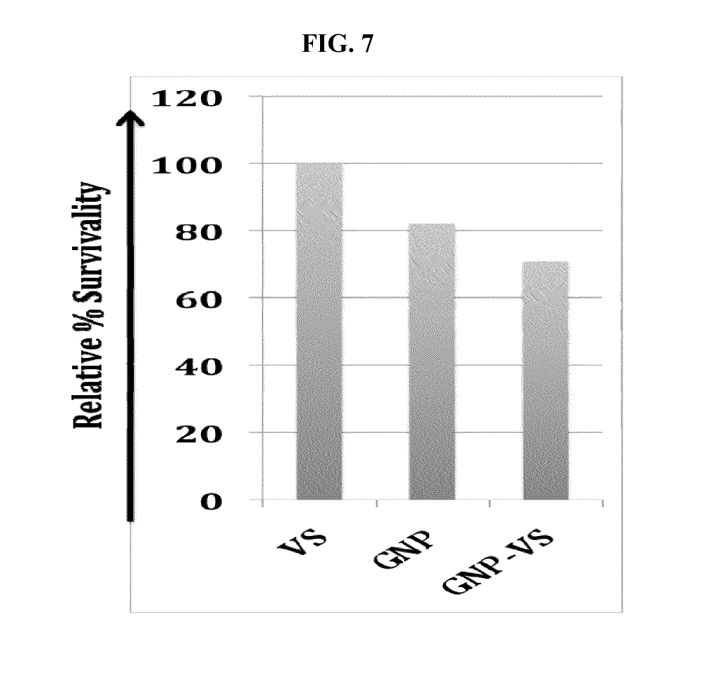

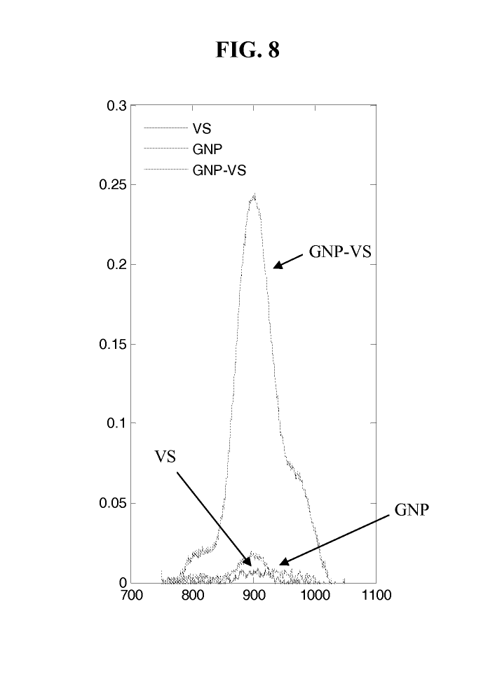

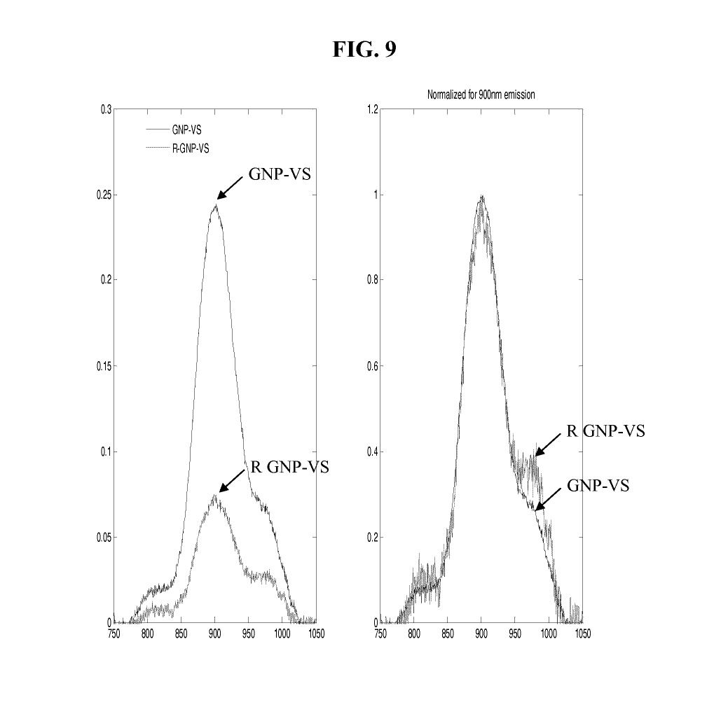

FIG. 7 shows the results of the standard MTT assay for cell viability. Results are shown as a percentage of cells surviving in VS. The data show that GNP-VS reduced cell viability by about 25%. FIG. 8 shows NIR emission spectra for RPMI 8226 cells treated with different agents (VS, GNP, and GNP-VS). These results indicate that each cell type has a characteristic NIR pattern. FIG. 9 shows the NIR emission spectra of GNP conjugated with VS compared to GNP conjugated with arginine and VS in the RPMI 8226 cells. The NIR emission alters at 900 and 960 nm. The normalized spectra shows that that there is a spectral change at 960 nm.

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 proteins refers to groups having 1, 2, or 3 proteins. Similarly, a group having 1-5 proteins refers to groups having 1, 2, 3, 4, or 5 proteins, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference in their entireties and for all purposes to the same extent as if each individual publication, patent, or patent application was specifically and individually incorporated by reference in its entirety for all purposes.

1. A method for identifying one or more cells in a sample, the method comprising:
passing a cell from the sample through a cell detection zone;
illuminating the cell in the cell detection zone with an effective amount of electromagnetic radiation to produce a near-infrared emission; and
analyzing an intensity and pattern of the near-infrared emission at about 900 to about 1000 nm to identify the cell in the cell detection zone.

2. The method of claim 1, wherein the electromagnetic radiation is produced by a laser.

3. The method of claim 2, wherein a wavelength of electromagnetic radiation produced by the laser is about 630±20 nm.

4. (canceled)

5. The method of claim 1, wherein the cell is a eukaryotic cell, a prokaryotic cell, an embryonic stem cell, or an adult stem cell.

6. The method of claim 5, wherein the cell is selected from the group consisting of: a red blood cell, a platelet, and a mononuclear cell.

7. (canceled)

8. (canceled)

9. The method of claim 5, wherein the cell is an embryonic stem cell.

10. The method of claim 5, wherein the cell is an adult stem cell.

11. The method of claim 5, wherein the cell is a hematopoietic stem cell.

12. The method of claim 1, wherein the sample comprises at least one cancer cell.

13. The method of claim 12, wherein the analyzing comprises detecting the at least one cancer cell.

14. The method of claim 13, wherein the analyzing comprises comparing the measured near-infrared emission profile to a near-infrared emission of a normal cell, or to a near-infrared emission of a cancer cell, or to both, in order to detect the at least one cancer cell.

15. The method of claim 1, wherein the sample contains or is suspected to contain at least one pathogen.

16. The method of claim 15, wherein the analyzing comprises detecting the at least one pathogen, if present.

17. The method of claim 1 further comprising detecting one or more additional label-free characteristics of the cell.

18. The method of claim 17, wherein the one or more additional label-free characteristics of the cell are selected from the group consisting of: forward scattering, side scattering, and pseudo-Raleigh scattering.

19. The method of claim 1, wherein passing the cell from the sample through the cell detection zone is by flow cytometry.

20. The method of claim 19 further comprising sorting the cell.

21. The method of claim 20, wherein sorting comprises removing cells that are not in a cell population of interest.

22. The method of claim 21, wherein the cells that are not in the cell population of interest are destroyed.

23. A method for enriching a population of a desired cell type, the method comprising:
introducing a heterogeneous mixture of cells into a flow stream;
passing each cell in the heterogenous mixture of cells through a cell detection zone;
illuminating the cell in the cell detection zone with an effective amount of electromagnetic radiation to produce a near-infrared emission in the cell detection zone; and
collecting the cells that have a substantially identical intensity or pattern of the near-infrared emission at about 900 to about 1000 nm to the desired cell type in order to produce an enriched population of cells.

24. (canceled)

25. (canceled)

26. (canceled)
